(19) United States
(12) Patent Application Publication
(10) Pub. No.: US 2019/0117060 A1
(43) Pub. Date: Apr. 25, 2019

(54) A PHOTO-REFRACTION DEVICE FOR IDENTIFYING AND DETERMINING REFRACTIVE DISORDERS OF AN EYE

(71) Applicant: FORUS HEALTH PVT. LTD., Bangalore, Karnataka (IN)

(72) Inventors: Prasanna HEGDE, Bangalore (IN); Bharath HEGDE, Bangalore (IN); Mahabaleswara R. BHATT, Bangalore (IN); Shyam Vasudeva RAO, Bangalore (IN)

(21) Appl. No.: 16/094,800

(22) PCT Filed: Jul. 19, 2016
(86) PCT No.: PCT/IB2016/054287

§ 371 Date: Oct. 18, 2018

(30) Foreign Application Priority Data
Apr. 18, 2016 (IN) 201641013451

(51) Int. Cl. A61B 3/103; A61B 3/113; A61B 3/14

(52) U.S. Cl. CPC A61B 3/103; A61B 3/14; A61B 3/113

(57) ABSTRACT

The present disclosure discloses a method for capturing images of an eye of an unresponsive subject using a photo-refraction device comprising a computing unit and an optical adapter. The method comprises acts of detecting by an image capturing unit face of the unresponsive subject, navigating by processing unit, the image capturing unit towards eye region of the unresponsive subject upon detecting face of the unresponsive subject. The method further comprises acts of capturing by the image capturing unit, images of the pupil in real time, analyzing by the processing unit, captured images of the pupil to determine feasibility of pupil position. The method also comprises acts of activating by the processing unit, a plurality of light sources configured in the optical adapter of the photo-refraction device for sequentially illuminating light on the pupil and capturing by the image capturing unit, images of the pupil during sequential illumination of light.

### TECHNICAL FIELD

The present invention generally relates to the field of biomedical devices. Particularly, but not exclusively, the present disclosure relates to device for identifying refractive disorders in an eye. Further embodiments of the present disclosure disclose a photo refraction device for identification and determining refractive disorders in an eye of an unresponsive subject.

### BACKGROUND OF THE DISCLOSURE

Generally, in refractive disorders, light rays entering eye of the subjects are not focused on the retina, which would cause blurred vision. Normally, the cornea and lens refract incoming light rays to focus them on the retina. When there is a refractive error, the cornea and lens cannot focus light rays on to the retina. The lens and cornea of the eye may not project light rays to focus them on the retina correctly for several reasons, which includes but are not limited to nearsightedness, farsightedness and astigmatism. These refractive errors may thus cause eye abnormalities leading to blindness progressively. Therefore, it is important that these refractive errors are detected at early stages itself and these refractive errors maybe corrected by eyeglasses, contact lenses or surgery.

Conventionally, to identify refractive errors in the eye of the subject or a patient either retinoscopy methods or auto-refraction methods are followed. Especially, in auto-refractometers a large area of the retina is illuminated and a camera is focused on the retina, to image details of its anatomy and the related images are later analyzed to identify errors. However, such conventional devices are bulky and therefore have limited applications. With the advent of modernization, many efforts have been made to develop compact devices for measuring refractive disorders. One of such devices includes photorefractive device which may be used to collect and analyze ocular responses to light stimuli. In such photorefractive devices, light from an external source enters the eye through the pupil and is focused to create a small illuminated spot on the retina. Some of the light from this retinal spot is returned out of the eye through the pupil after interaction with different layers of the retina. The pattern of light exiting the pupil is determined by the optics of the eye and is dominated by an examinee's refractive error (focusing errors of the eye). The photo-refraction does not directly image the retina or any other structures in the posterior segment of the eye. In photo-refraction, images are obtained by focusing on the pupil to obtain the light pattern exiting the pupil—i.e., images are analyzed in the pupil plane.

Further, the existing arts for diagnosing eye of a patient are vastly based on the subjective assessment and hence screening may necessitate expert user and patient interaction which limits the scope of usage of such devices in public screening. Although there are mobile screener devices, which are portable standalone devices using photo-refraction principle in an objective approach where patient expert direct involvement is absent, it is a complete system having large number of optical components and camera set up, associated computer systems. Thus, it is not affordable and also presence of large number of components makes the system complex. In addition, public screening practitioner is not facilitated with a provision of utilizing such accessory. Further, the conventional techniques find the axis, cylindrical error, spherical error mainly by approximation and complex calibration methods which would have limitations such as calibration becomes complex and finding the parameters of refraction error are complex and inefficient. Furthermore, in the conventional screening methods for identification and determining refractive disorders, the subject or a patient is required to concentrate for long durations and the same cannot be applied during screening of unresponsive subjects such as an infant or a child.

In light of the foregoing discussion, there is a need to develop an improved photo-refraction device to overcome one or more limitations stated above.

### SUMMARY OF THE DISCLOSURE

One or more shortcomings of the prior art are overcome and additional advantages are provided through the present disclosure. Additional features and advantages are realized through the techniques of the present disclosure. Other embodiments and aspects of the disclosure are described in detail herein and are considered a part of the claimed disclosure.

In one non-limiting embodiment of the disclosure, there is provided a method for capturing images of an eye of an unresponsive subject using a photo-refraction device comprising a computing unit and an optical adapter. The method comprises acts of detecting by an image capturing unit configured in the computing unit of the photo-refraction device face of the unresponsive subject, navigating by processing unit of the computing unit the image capturing unit towards eye region of the unresponsive subject upon detecting face of the unresponsive subject. The method further comprises acts of capturing by the image capturing unit, images of the pupil in real time upon identifying the eye region of the unresponsive subject, and analyzing by the processing unit, captured images of the pupil to determine feasibility of pupil position.

The method also comprises acts of activating by the processing unit, a plurality of light sources configured in the optical adapter of the photo-refraction device for sequentially illuminating light on the pupil upon determining the feasibility of pupil position and capturing by the image capturing unit, images of the pupil during sequential illumination of light.

In an embodiment of the disclosure, the method comprises an act of controlling by the computing unit, sequence of illumination of the plurality of light sources. Further, the plurality of light sources is array of Light Emitting Diodes (LEDs).

In an embodiment of the disclosure, the method comprises an act of receiving by an optical unit configured in the optical adapter, irradiant rays of light from the pupil during sequential illumination of light. The optical unit is configured to direct the irradiant rays of light from the pupil onto the image capturing unit. Further, the image capturing unit captures the images of the irradiant rays of light from the pupil.

In an embodiment of the disclosure, the method comprises an act of analyzing by the computing unit, captured images of the irradiant rays of light from the pupil to determine refractive disorder parameters.

In an embodiment of the disclosure, the method comprises an act of communicating, by the computing unit, the refractive disorder to an external device.

In another non-limiting embodiment of the disclosure, there is provided a photo-refraction device for capturing images of an eye of an unresponsive subject. The device comprises an optical adapter comprising a plurality of light sources and an optical unit and a computing unit. The computing unit comprises an image capturing unit, a processing unit and a memory unit and is interfaced with the optical adapter. The processing unit is configured to detect face of the unresponsive subject via the image capturing unit, navigate the image capturing unit towards eye region of the unresponsive subject upon detecting face of the unresponsive subject. The processing unit is also configured to receive images of the pupil captured in real time by the image capturing unit upon identifying the eye region of the unresponsive subject, analyze received images of the pupil to determine feasibility of pupil position. The processing unit is also configured to activate a plurality of light sources configured in the optical adapter for sequentially illuminating light on the pupil upon determining the feasibility of pupil position, and further activate the image capturing unit to capture images of the pupil during sequential illumination of light.

In an embodiment of the disclosure, the computing unit comprises a power source to supply power to the plurality of LED arrays.

In an embodiment of the disclosure, the processing unit is configured to process and analyze images obtained by irradiant rays of light from the pupil for measurement of refractive disorder parameters.

In an embodiment of the disclosure, the memory unit is configured to store captured images of the pupil.

In another non-limiting embodiment of the disclosure, there is provided a method for determining refractive disorder parameters of an eye of an unresponsive subject. The method comprises the act of extracting, by a photo-refraction device, plurality of pixels forming pupil portion of the eye from an image captured using an image capturing device, wherein the plurality of pixels forming the pupil portion of the eye form a feature set. The method comprises obtaining a gradient image of the pupil portion of the eye by determining direction of each of the plurality of pixels in the feature set. Further, the method comprises aligning the one or more pixels having similar direction to form one or more lines in the gradient image. The method also comprises identifying a best-fit-line among the one or more lines in the gradient image for obtaining a slope parameter corresponding to the identified best-fit-line. Finally, the method assigns the slope parameter as the refractive disorder parameter along a meridian axis corresponding to one of a plurality of light sources associated with the image capturing device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The disclosure itself, however, as well as a preferred mode of use, further objectives and advantages thereof, will best be understood by reference to the following detailed description of an illustrative embodiment when read in conjunction with the accompanying figures. One or more embodiments are now described, by way of example only, with reference to the accompanying figures wherein like reference numerals represent like elements and in which:

FIG. 1 illustrates a flowchart depicting a method for capturing images of an eye of an unresponsive subject using a photo-refraction device, according to some embodiment of the present disclosure.

FIG. 2 illustrates a block diagram of the photo-refraction device used for capturing images of the pupil using the method of FIG. 1 and for determining refractive disorders of the eye of an unresponsive subject, according to some embodiment of the present disclosure.

FIG. 3 illustrates a schematic diagram of a geometrical arrangement of the photo-refraction device for capturing images of an eye of an unresponsive subject using the method of FIG. 1, according to some embodiment of the present disclosure.

FIGS. 4A-4C illustrates schematic representations of light sources configured in the device of FIG. 2 for sequentially illuminating light on to pupil of an unresponsive subject, according to some embodiment of the present disclosure.

FIG. 5 illustrates a flowchart showing a method for determining refractive disorder parameters of an eye of an unresponsive subject in accordance with some embodiments of the present disclosure.

FIG. 6A and FIG. 6B show graphical representation of the meridian refractive disorder parameter in accordance with some embodiments of the present disclosure.

FIG. 7 illustrates a flowchart showing an exemplary method for calibrating the spherical disorder parameter of an eye of an unresponsive object in accordance with some embodiments of the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

To overcome one or more limitations stated in the background, the present disclosure discloses a method for identifying refractive disorders of an eye in an unresponsive subject using a photo-refraction device. The method of the present disclosure is useful to identify refractive disorders of an unresponsive subjects including but not limited to infants or human beings who find difficulty in concentrating during photo-refraction screening. In an embodiment of the disclosure, principle adopted to capture images of an unresponsive eye for identifying refractive disorders is eccentric photo refraction. The refractive disorders which may be identified and determined according to the present disclosure includes but are not limited to axis, cylindrical and spherical errors, anysometropia etc. By adopting the method of present disclosure, a medical practitioner will be able to perform eccentric photo refraction screening to identify refractive disorders or eye abnormalities without giving any verbal instructions to diagnose a subject such as infants.

As described in the previous paragraph, the method aims to capture images of an eye of the unresponsive subject for identification of refractive disorders (if any) using the photo-refractive device comprising computing unit and optical adapter. In an embodiment, the computing unit is at least one of smartphone and a Personal Digital Assistant (PDA), and the image capturing unit is a camera of the smartphone or PDA. The computing unit such as smartphone or PDA comprises a processing unit to control and support the operations of computing unit. The method comprises steps of—firstly, an image capturing unit of the computing unit detects face of the unresponsive subject. Upon detection of face of the unresponsive subject, the processing unit navigates the image capturing unit or the camera towards eye region of the responsive subject and thereby navigates of the image capturing unit towards pupil of the eye. In an embodiment, if navigation of the image capturing unit towards eye region fails, the photo-refraction device reverts to the initial stage and then continues with detection of face and so on. Subsequently, the image capturing unit of the computing unit is directed to capture a plurality of images of the pupil upon detection of eye and pupil region of the unresponsive subject. After capturing real time images of the pupil, the processing unit of the computing unit analyzes these captured images of the pupil to determine feasibility of the captured region of the pupil for further processing and identification of the refractive disorders. In an embodiment of the disclosure, if the captured pupil region of the eye of the unresponsive subject is not feasible for further processing, the method goes back to stage of detection of face and so on. Once, a feasible region of the pupil is identified by the processing unit, the processing unit activates a plurality of light sources configured in the optical adapter of the photo-refraction device for sequential illumination of light on the pupil of the eye. In an embodiment of the disclosure, the plurality of light sources is an array of Light Emitting Diodes (LEDs) and emits infrared rays. Further, during sequential illumination of light on the pupil region of the eye, the image capturing unit of the computing unit captures plurality of images of the pupil for further analysis by the processing unit of the computing unit to identify refractive disorders. In an embodiment of the disclosure, the computing unit controls the functioning of LED light source i.e. the illumination, time period for which the light source is switched on, amount of power required etc is controlled by the computing unit. The computing unit also supplies the necessary power for illumination by the LED light source.

Further, the optical adapter of the photo-refraction device comprises an optical unit for receiving the irradiant rays of the light from the pupil and directing it onto the computing unit for further analysis. In an embodiment of the disclosure, the optical adapter may be interfaced with any smartphones or PDAs without any need for additional light sources, processing unit, memory unit etc.

In the following detailed description of the embodiments of the disclosure, reference is made to the accompanying drawings that form a part hereof, and in which are shown by way of illustrating specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

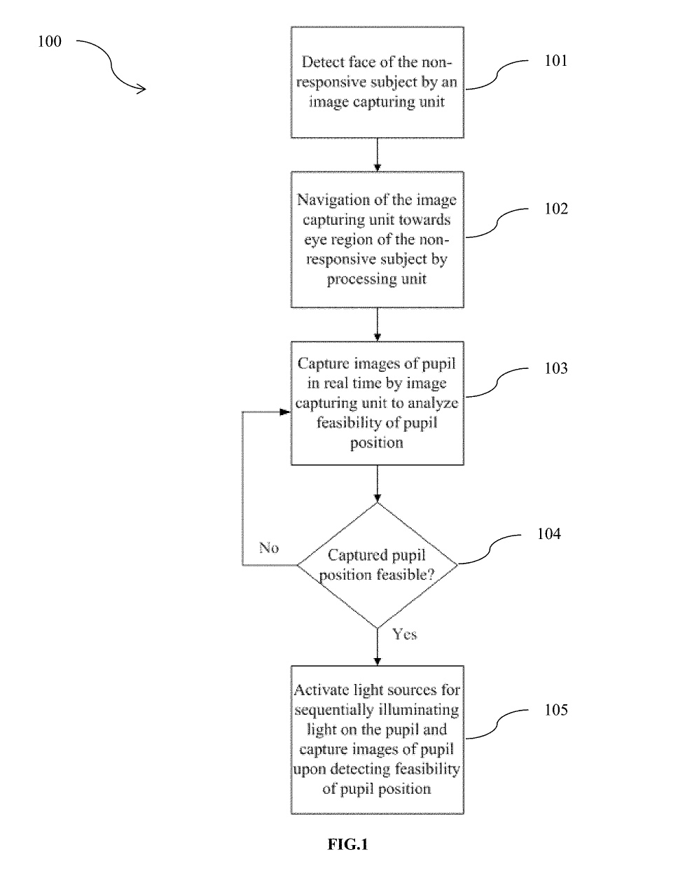

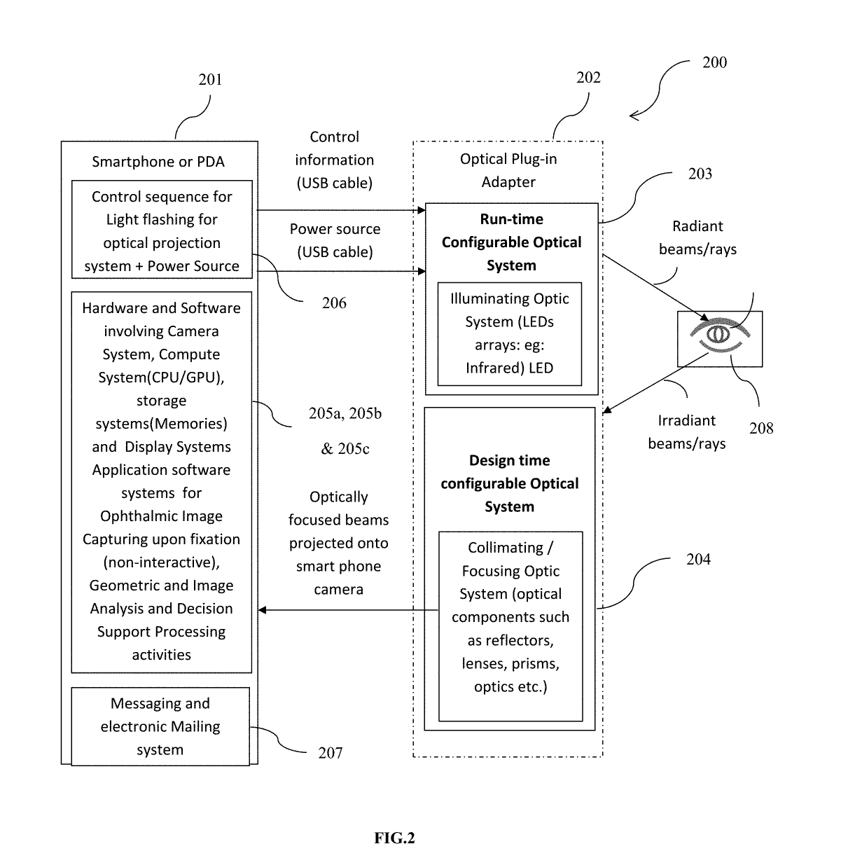

FIG. 1 is an exemplary embodiment of the present disclosure which illustrates a flowchart depicting a method for capturing images of an eye of an unresponsive subject using a photo-refraction device. As illustrated in FIG. 1, the method comprises one or more blocks for capturing images of an eye of an unresponsive subject using an apparatus as illustrated in FIG. 2.

The method may be described in the general context of computer executable instructions. Generally, computer executable instructions can include routines, programs, objects, components, data structures, procedures, modules, and functions, which perform particular functions or implement particular abstract data types.

The order in which the method 100 is described is not intended to be construed as a limitation, and any number of the described method blocks can be combined in any order to implement the method. Additionally, individual blocks may be deleted from the methods without departing from scope of the subject matter described herein. Furthermore, the method can be implemented in any suitable hardware, software, firmware, or combination thereof.

The method as disclosed in FIG. 1 is useful for identifying refractive disorders in unresponsive subjects including but not limited to infants and human beings who find difficulty in concentrating during photo-refraction screening. The principle adopted to capture images of a unresponsive eye for identifying refractive disorders is eccentric photo refraction. The method of the present disclosure to capture images of the pupil and identifying refractive disorders is now described with reference to the flowchart blocks and is as below—

At block 101, the method comprises an act of detecting face of the unresponsive subject. The method adopts a photo-refraction device (200) which comprises a computing unit (201) and an optical adapter (202). In an embodiment of the disclosure, the computing unit (201) is a smartphone or a PDA (Personal Digital Assistant). The face is detected by an image capturing unit (205a) of the computing unit (201). In an embodiment of the disclosure, the image capturing unit (205a) is a smartphone camera or a camera of the PDA. It is to be understood that the present disclosure is not limited to any particular smartphone or PDA and hence any handheld device having image capturing unit which serve the purpose may be considered. The photo-refraction device (200) is positioned at a pre-determined distance from a subject to carry out its operation.

At block 102, upon detection of the face of the unresponsive subject, the image capturing unit (205a) such as smartphone camera or PDA camera is navigated towards the eye region (208) of the unresponsive subject. Since, the method particularly focuses on unresponsive subjects, verbal communication between the subjects or patient and the medical practitioner may be avoided, and the processing unit (205b) of the smartphone or PDA navigates the camera towards eye region of the subject. For identification of refractive disorders, pupil region (208) of the eye is then focused and hence image capturing unit (205a) is navigated towards the anterior region of the pupil. In an embodiment of the disclosure, if the image capturing unit (205a) is not successfully navigated towards the eye region (208), the method reverts to the act of detection of face of the unresponsive subject as shown in block 101.

At block 103, once the image capturing unit (205a) is navigated towards a particular position of the pupil region of the eye (208), images of the pupil are captured with the aid of image capturing unit (205a). The image capturing unit (205a) i.e. the smartphone camera or PDA camera captures live images or real time images of the pupil of the unresponsive subject. Upon capturing images of the pupil at the said position, the captured images of the pupil are processed by the processing unit (205b) of the computing unit (201). The captured images are analyzed by the processing unit (205b) to check the feasibility of the pupil position i.e. the processing unit (205b) analyzes the captured images to check if the pupil position is appropriate for identification and determining of the refractive disorders [as shown in block 104 of FIG. 1]. The real time images of pupil is analyzed if the pupil position assessed is feasible to identify required refractive disorder parameters like gradient without having over or under exposures in the pupil area. If the captured images of the pupil position are not feasible for identification and determining of the refractive disorders, the image capturing unit (205a) captures fresh images of the pupil now at a different position. Upon capturing fresh images of the pupil, the processing unit (205b) again analyzes the feasibility of the pupil position and this continues till a feasible position of the pupil for identification and determining of the refractive disorders is found out.

At block 105, after a feasible pupil position for further processing is identified, the processing unit (205b) activates a plurality of light sources (203) for sequentially illuminating light on the pupil. In an embodiment of the disclosure, the plurality of light sources (203) is an array of Light Emitting Diodes (LEDs) emitting Infrared (IR) rays. Further, the plurality of LED light sources (203) is configured in the optical adapter (202) of the photo-refraction device (200) and the LED light sources (203) may be controlled by the computing unit (201). In an embodiment, the LED light sources (203) is controlled on the basis of intensity of light, sequence of illumination, optical projection etc. The power required for illumination of light on the pupil from the light sources (203) is supplied by a power source (206) configured in the computing unit (201). In an embodiment of the disclosure, the power source (206) is a smartphone battery or a PDA battery. In an embodiment of the disclosure, required power for illumination of light sources (203) is supplied through a USB cable and in another embodiment, illumination of light sources is controlled by a control information USB cable. Furthermore, at block 105, images of the pupil are captured during sequential illumination of light by the LED light sources (203). The images of the pupil are captured and irradiated towards the computing unit (201) of the photo-refraction device (200) for identifying refractive disorders and thereafter for determining the refractive disorder parameters. Further, the images captured maybe transferred to an external device for further processing and analysis to determine refractive disorders in the eye. In an embodiment of the disclosure, unresponsive subject is a subject who is not responsive to commands. For example, unresponsive subject includes infants, lunatics, person with hearing disability etc.

Referring now to FIG. 2, which is an exemplary embodiment of the present disclosure, illustrates a block diagram of the photo-refraction device (200) for capturing images of the pupil for determining refractive disorders of the eye (208) of an unresponsive subject. The photo-refraction device (200) mainly comprises of two sub systems—computing unit (201) and optical adapter (202). In an embodiment of the disclosure, the optical adapter (202) may be interfaced with the computing unit (201) in any known forms such as optical adapter (202) may be plugged to the computing unit (201).

The optical adapter (202) of the photo-refraction device (200) is further classified into run-time configurable optical system and design time configurable optical system. The optical function configurable at run-time optical system is an array of LED light sources (203) for sequential illumination of light on the pupil of eye (208). In an embodiment of the disclosure, the LED light source is an Infrared (IR) LED light source. The optical function configurable at design time optical system is an optical unit (204) consisting of a plurality of collimating or focusing optic system such as, but not limited to, reflectors, lenses, prisms, optics.

The computing unit (201) of the photo-refraction device (200) comprises several hardware components with integrated software. In an embodiment, hardware and software components of the computing unit (201) includes power source (206), image capturing unit (205a) or camera, processing unit (CPU/GPU) (205b), storage system or memory unit (205c), display systems, application software systems for ophthalmic image capturing upon fixation (non-interactive), geometric and image analysis and decision support processing activities and messaging or electronic mailing system (207). In an embodiment of the present disclosure, the computing unit (201) is a smartphone or a PDA (Personal Digital Assistant). The computing unit (201) also has control sequence for light flashing for optical projection system.

In an embodiment of the present disclosure, to capture plurality of images of the pupil of the unresponsive subject, IR LED light sources (203) sequentially illuminate the pupil region of eye (208) after performing functions such as face detection, navigation of the image capturing unit (205a) for pupil detection, capturing pupil position by the image capturing unit (205a) and analyzing feasibility of the pupil position for further processing. Sequential illumination of light sources is done by radiating beams/rays on the feasible region of the pupil. The rays radiated, are reflected and irradiant rays from the eye (208) are focussed onto the sensor of the image capturing unit (205a) of the computing unit (201). In an embodiment of the disclosure, the irradiant rays from the eye (208) are focussed onto the camera sensor of the smartphone or PDA. Upon receipt of the irradiant rays from the pupil by the image capturing unit (205a) of the computing unit (201), the captured images of the eye are stored in a memory unit (205c). Thereafter, the processing unit (205b) processes the images captured by accessing the memory unit (205c) to identify the refractive disorders of the eye. In an embodiment of the disclosure, for capturing images of the pupil of an unresponsive subject such as an infant or child, the photo-refraction device (200) may be configured as part of an attractive exhibit or children gaming device that invariably draws attention of the child or infant for a noticeably longer duration. In an embodiment of the disclosure, the computing unit (201) comprises a messaging or mailing system (207) to communicate various necessary data and information to the expert or health archive or user, in any combinations.

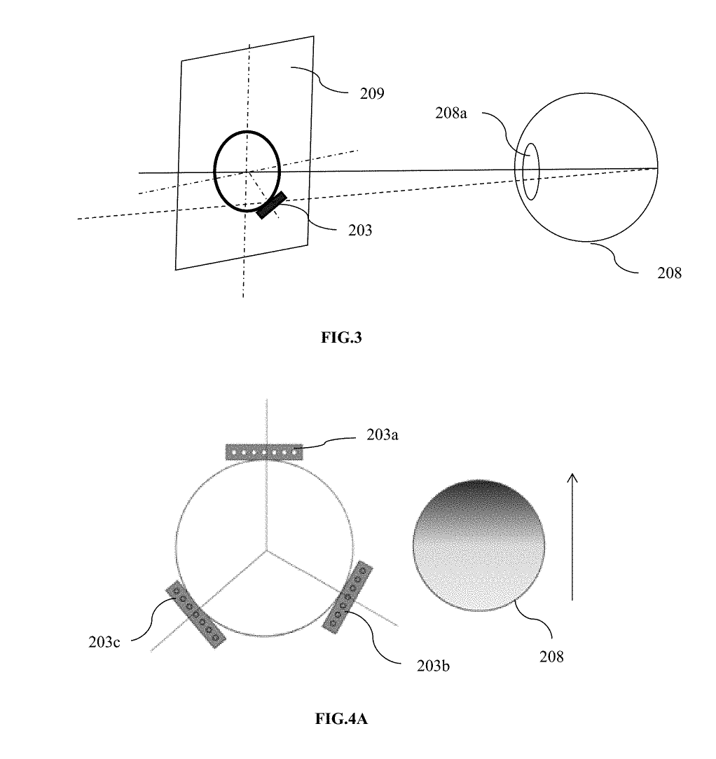

Referring now to FIG. 3, which is an exemplary embodiment of the present disclosure, illustrates a geometrical arrangement of the optical setup for capturing images of the eye (208) of unresponsive subject. The optical setup comprises LED illumination light sources (203) that emit IR rays and an image plane (209) capable of capturing images of the pupil (208a) of the eye (208). The images of the pupil (208a) are captured by the method as disclosed in the previous paragraphs. The photo-refraction screening of the present disclosure uses the principle of eccentric photo-refraction and hence the light sources (203) are positioned at angle with the horizontal meridian. Furthermore, the image plane (209) of the optical setup comprises an image sensor [not shown in the figure]. The image sensor of the image plane (209) is configured to receive the irradiated rays from the pupil (208a) of the eye (208) after illumination of light on the pupil (208a) by the plurality of light sources (203). In an embodiment of the disclosure, the image sensor is typically positioned at a distance of about 1 meter to 1.5 meters from the non-responsive subject. In addition, as described in the previous paragraphs, the sequence of illumination of the IR LED light sources (203) can be controlled by the computing unit (201) of the photo-refraction device (200).

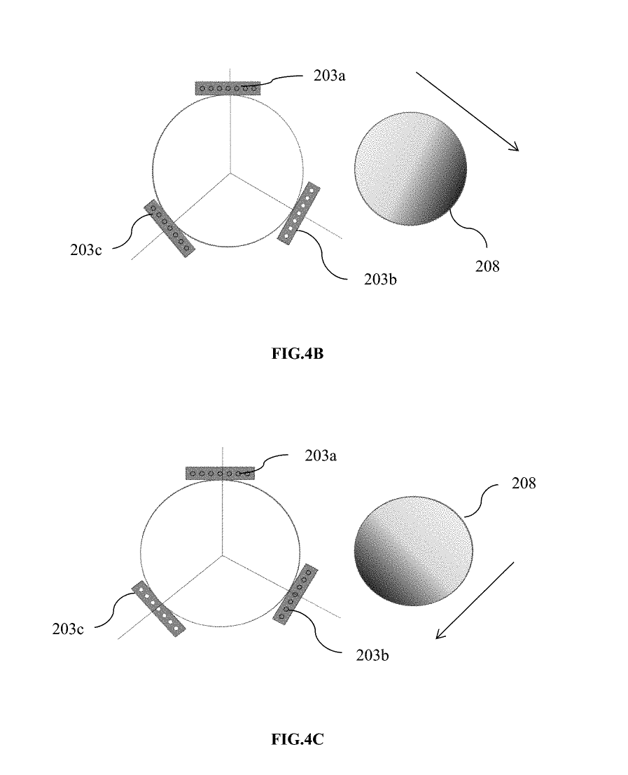

FIG. 4A, FIG. 4B and FIG. 4C are exemplary embodiments of the present disclosure which illustrates schematic representations of sequential illumination of light sources (203) for capturing images of pupil of an unresponsive subject. In an embodiment of the disclosure, the plurality of light sources are three in number including first, second, third LED light sources as shown in FIGS. 4A-4C. However, the number of light sources should not be considered as limitation, as the method may be extended to any number LED light sources.

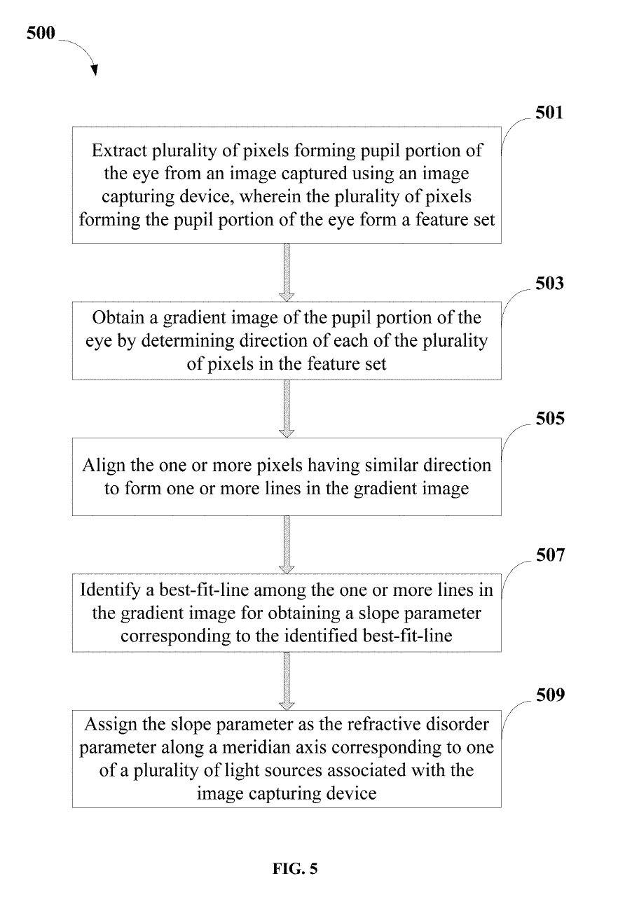

Referring to FIG. 4A which is an exemplary embodiment of the disclosure, in which images of the pupil of the eye (208) is captured during illumination of the first LED light source (203a). As shown in FIG. 4A second and third LED light sources (203b and 203c) are in OFF position. In an embodiment, during illumination of the first LED light source (203a) in the meridian of degrees θ(0), refractive disorder R[θ(0)] is computed by evaluating the slope or gradient of the intensity of the pupil image in the direction as shown in FIG. 4A. The method for computation of refractive disorder parameters will be described in conjunction with FIG. 5.

FIG. 4B is an exemplary embodiment of the disclosure, in which images of the pupil of the eye (208) is captured during illumination of the second LED light source (203b), whereas the first and third LED light sources (203a and 203c) are in OFF position. In an embodiment, during illumination of the second LED light source (203b) in the meridian of degrees θ(π/3), refractive disorder R[θ(π/3)] is computed by evaluating the slope or gradient of the intensity of the pupil image in the direction as shown in FIG. 4B. The method for computation of refractive disorder parameters will be described in subsequent figures in conjunction with FIG. 5.

FIG. 4C is an exemplary embodiment of the disclosure, in which images of the pupil of the eye (208) is captured during illumination of the third LED light source (203c), whereas the first and second LED light sources (203a and 203b) are in OFF position. In an embodiment, during illumination of the third LED light source (203c) in the meridian of degrees θ(2π/3), refractive disorder R[θ(2π/3)] is computed by evaluating the slope or gradient of the intensity of the pupil image in the direction as shown in FIG. 4C. The method for computation of refractive disorder parameters will be described in subsequent figures in conjunction with FIG. 5.

FIG. 5 illustrates a flowchart showing a method for determining refractive disorder parameters of an eye of an unresponsive subject in accordance with some embodiments of the present disclosure.

At block 501, a photo-refraction device (200) extracts plurality of pixels forming pupil (208a) portion of the eye (208) from an image captured using an image capturing unit (205a). The plurality of pixels forming the pupil (208a) portion of the eye (208) form a feature set comprising the plurality of pixels. In an embodiment, the pupil (208a) portion of the eye (208) may be extracted using an image processing and analysis technique. Further, one or more redundant pixels in the feature set may be eliminated in order to obtain an effective feature set.

At block 503, the photo-refraction device (200) obtains a gradient image of the pupil (208a) portion of the eye (208) by determining direction of each of the plurality of pixels in the feature set. The direction of each of the plurality of pixels is one of a vertical direction along the obtained image gradient image. In an embodiment, the complete gradient image of the pupil (208a) portion of the eye (208) may be obtained using a two-step process. The first step comprises obtaining the gradient image of the extracted pupil (208a) portion of the eye (208). The second step comprises aligning the obtained gradient image along the direction of the light sources (203) by rotating the gradient image in a predefined direction in an increasing and/or decreasing order of grey-scale pixel variation. As an example, the predefined direction for rotating the gradient image may be at least one of clockwise direction and anti-clockwise direction.

At block 505, the photo-refraction device (200) aligns the one or more pixels having similar direction to form one or more lines in the gradient image. The direction of each of the plurality of pixels is determined by measuring an angle of inclination of each of the plurality of pixels with a reference co-ordinate axis, say, X co-ordinate axis. Further, the one or more pixels having similar direction align themselves to form one or more horizontal line on the obtained gradient image.

At block 507, the photo-refraction device (200) identifies a best-fit-line among the one or more lines in the gradient image for obtaining a slope parameter corresponding to the identified best-fit-line. In an embodiment, the best-fit-line is the line joining plurality of correlating pixels among the plurality of pixels in the feature set. A general strategy used for identifying the best-fit line in the gradient image is based on “Least deviation” associated with each of the one or more horizontal lines, as explained below.

### General Strategy: Least Deviation Based Best-Fit-Line Identification:

A horizontal line with the least deviation (represented by l_{q}, 0<q<∞) may be considered as the best-fit line when the line l_{q} is laid in the graph of the function {a*p_{i,j}+b*} where: a* and b*∈R. Now, the slope parameters of the line l_{q} are determined by performing minimization on a and b as shown in equation (1) below:

$$f ( a , b ) = {min}_{a , b} {\{ {\sum }_{i = 1}^{N} {w}_{i} {\vert {y}_{i} - ( {ap}_{i , j} + b ) \vert}^{q} \}}^{1 / q} \qquad ( 1 )$$

Where, {p_{i,j}, i=1, 2, . . . , N for selected j^{th} line in the given image}, N>2, and wᵢ>0 correspond to the line l_{q}.

In an embodiment, the method and implementation of identifying the best-fit line from the one or more horizontal lines may use various other best-fit line strategies (Strategy 1 to Strategy 3) that are described in detail in the below section. However, each of the best-fit line strategies (Strategy 1 to Strategy 3) may use the same general strategy to identify the best-fit line.

### Strategy 1: Least Absolute Deviation Based Best-Fit-Line Identification.

The Strategy 1 may be implemented in an alternative embodiment to the General strategy. Here, the best-fit line l₁ is a line that best fits into the graph of the function a*p_{i,j}+b*, where a*, b*∈R whose slope parameters are determined by performing minimization on a and b using the equation (2) below:

$$f ( a , b ) = {min}_{a , b} \{ {\sum }_{i = 1}^{N} {w}_{i} {\vert {y}_{i} - ( {ap}_{i , j} + b ) \vert}^{1} \} \qquad ( 2 )$$

Where, {p_{i,j}, i=1, 2, . . . , N for selected j^{th} line in the given image}, N>2, and wᵢ>0 are correspond to the line l₁.

### Strategy 2: Least Squares Deviation Based Best-Fit-Line Identification:

The Strategy 2, similar to the Strategy 1, may be implemented in another alternative embodiment to the General strategy. In an embodiment, considering that the best-fit line comprises a slope a and an Intercept b. Now, consider a data array having the intensity or pixel values for a j^{th} selected horizontal line of the gradient image of the pupil (208a). The data array may be formed by selecting any j^{th} row; preferably a row from a mid-row of the gradient image and designating the j^{th} row as a set T={(i, wᵢ×p_{i,j}), i=1, 2, . . . , N,}.

The initial parameters for the selected line j^{th} are computed using the below sequence of steps:

$${i}_{o} = {\sum }_{i = 1}^{N} i$$

$${p}_{o , j} = {\sum }_{i = 1}^{N} {p}_{i , j}$$

$${a}_{0} = \frac{1}{{\sum }_{i = 0}^{N} {( i - {i}_{o} )}^{2}} \{ {\sum }_{i = 1}^{N} ( i - {i}_{o} ) ( {p}_{i , j} - {p}_{0 , j} ) \}$$

$${b}_{0} = {p}_{{i}_{0} , j} - {a}_{o} {i}_{0}$$

Similarly the weight element on the best-fit line may be determined using the equation (3) below:

$${ω}_{i} = \frac{1}{\vert {p}_{i , j} - {a}_{0} i - {b}_{0} \vert} , i = 1 , 2 , … , N \qquad ( 3 )$$

The equation (3) may be again used to compute centroids of the best-fit line using the below equations.

$$ω = {\sum }_{i = 1}^{N} {ω}_{i} , \\ {i}_{0} = {\sum }_{i = 1}^{N} ( {ω}_{i} \times {p}_{i , j} )$$

$${p}_{o , j} = \frac{1}{ω} {\sum }_{i = 1}^{N} ( {ω}_{i} \times {p}_{i , j} )$$

Then the slope parameters a₁ and b₁ are computed using the equation (4) and (5) below:

$$\begin{matrix}{a}_{1} = \frac{1}{{\sum }_{i = 0}^{N} {ω ( i - {i}_{o , j} )}^{2}} \{ {\sum }_{i = 1}^{N} {ω}_{i} ( i - {i}_{o} ) ( {p}_{i , j} - {p}_{0 , j} ) \} & ( 4 ) \\ {b}_{1} = {p}_{p , j} - {a}_{1} {i}_{0} & ( 5 )\end{matrix}$$

Further, the method of calculating the weight elements is repeated by setting a₀=a₁, b₀=b₁, while a maximum value of the weight element, among the ωᵢ, i=1, 2 . . . N is less than N. Otherwise, the slope is determined to be the earlier weight element of the line when ωᵢ, i=1, 2 . . . N is higher than N.

Strategy 3: Least l_{∞} (Chebyshev) Deviation Based Best-Fit-Line:

The Strategy 3, similar to the Strategies 1 and 2, may be implemented as another alternative embodiment to the General strategy. The least l_{c} deviation or Chebyshev based best-fit line fits into a graph defined by the function a*p_{i,j}+b* where a*, b*∈R and whose slope parameters are determined by performing minimizations using the equation (6) below:

$$f ( a , b ) = {min}_{a , b} \{ {max}_{{p}_{i , j}} {w}_{i} \vert {y}_{i} - ( {ap}_{i , j} + b ) \vert \} \qquad ( 6 )$$

Where, {p_{i,j}, i=1, 2, . . . , N for the selected j^{th} line in the given image}, N>2, and wᵢ>0 are the corresponding weights for the line l_{∞}.

In an embodiment, the Strategy 3 comprises finding the slope-intercept parameters for the j^{th} row selected form the gradient image of the pupil (208a) of the eye (208). An arbitrary row of pixels is selected from the gradient image along with a weighting coefficient wᵢ (typically wᵢ>0) corresponding to each of the plurality of pixels. A set of pixels {(i, wᵢ×p_{i,j},), i=1, 2, . . . , N, for any j^{th} row} are identified. As an example, the j^{th} row may be a row that is belongs to the middle portion of the gradient image. Further, the centroid data point (i₀, pᵢ_{₀}_{,j})∈I may be identified using the equations below:

$${p}_{0 , j} = \frac{1}{N} {\sum }_{i = N}^{N} {p}_{i , j}$$

$${i}_{0} = \frac{( N - 1 )}{2}$$

Furthermore, two data points P₁(p_{Max1,j}, i_{P}_{₁}) & P₂(p_{Max2,j}, i_{P}_{₂})∈T are selected based on the following expressions:

$${p}_{Ma x 1 , j} = {max}_{1 \leq i \leq N} \{ {p}_{i , j} \}$$

$${p}_{Ma x 2 , j} = {max}_{{1 \leq i \leq N}_{{y}_{i} \neq {y}_{M ax 1}}} \{ {p}_{i , j} \}$$

Now, upon selecting p_{Max1,j} and p_{Max2,j}, an associated pixel co-ordinate location of the j^{th} row is identified as i_{P}_{₁} and i_{P}_{₂}. Furthermore, the slope parameters for the straight line p_{i,j}=a_{U}i+b_{U} are determined by passing the straight line through the points P₁(p_{Max1,j}, i_{P}_{₁}) & P₂(p_{Max2,j}, i_{P}_{₂})∈T. Then, the slope intercept of the straight line is computed using the equations (8) and (9) below:

$$\begin{matrix}{a}_{U} = ( \frac{{p}_{Ma x 1 , j} - {p}_{Ma x 2 , j}}{{i}_{{P}_{1}} - {i}_{{P}_{2}}} ) & ( 8 ) \\ {b}_{U} = ( {a}_{U} {i}_{{P}_{1}} + {p}_{{i}_{1} , j ,} ) & ( 9 )\end{matrix}$$

In a similar way, the Strategy 3 may be further used to identify a straight line passing through the point P₀(p_{0,j}, i_{P}_{₀}) and parallel to the above obtained straight line p_{i,j}=a_{U}i+b_{U} using the equation (10) given below:

b_{c}=pᵢ_{₀}_{,j},−(a_{U}i_{P}_{₀}+b_{U}) (10)

As a final step, the slope parameters for the straight line p_{i,j}=a_{U}i+b_{U} passing through the points P₁(p_{Max1,j}, i_{P}_{₁}) & P₂(p_{Max2,j}, i_{P}_{₂})∈T is determined using the equation (11) and (12) below:

$$\begin{matrix}{a}_{L} = ( \frac{{p}_{M i n 1 , j - {p}_{M i n 2 , j}}}{{i}_{{P}_{1}} - {i}_{{P}_{2}}} ) & ( 11 ) \\ {b}_{L} = ( {a}_{L} {i}_{{P}_{1}} + {p}_{{i}_{1} , j ,} ) & ( 12 )\end{matrix}$$

At block 509, the photo-refraction device (200) assigns the identified slope parameter as the refractive disorder parameter along a meridian axis corresponding to one of a plurality of light sources (203) associated with the image capturing unit (205a). In an embodiment, the slope parameter corresponding to the best-fit-line may be obtained by identifying plurality of points on the best-fit-line and determining a vertical change and a horizontal change in direction of each of the plurality of points on the best-fit-line. Further, a ratio of difference in vertical change of two of the plurality of points with difference in horizontal change of corresponding two of the plurality of points may be determined for identifying the slope-parameter of the best line.

In an embodiment, the refractive disorder parameters, such as spherical disorder parameter, axis disorder parameter and cylindrical disorder parameter, may be calculated using the best-fit line identified using one of the three strategies (Strategy 1-Strategy 3) explained above. The calculated refractive disorder parameters may be further used to determine one or more refractive errors in the unresponsive eye of an object (208).

In an embodiment, the photo-refraction device (200) forms a data array using refractive disorder parameters R_{θ(0)},

$${R}_{θ ( \frac{π}{3} )} and {R}_{θ ( \frac{2 π}{3} )}$$

in a predefined order of angle values. As an example, the predefined order of angle values for arranging the refractive disorder parameters may be increasing and/or decreasing order of angle values by considering one of the angle values as a reference angle in the range 0 and π along the meridian axis. Further, the data array may be extended by duplicating the formed data array along each of the angle values to get the symmetrically opposite located axes of meridians.

In an embodiment, upon extending the data array, the photo-refraction device (200) determines a Discrete Fourier transform (DFT) vector of the data array. A first element and a third element in the DFT vectors of the data array, which are analogous to a DC component and a second harmonic component of the DFT vector, are used to compute the spherical disorder parameter, the axis disorder parameter and the cylindrical disorder parameter.

The axis disorder parameter (φ) may be calculated as a Fourier phase transformation of the second harmonic component of the DFT vector as shown in below equation (13).

$$ϕ = {tan}^{- 1} ( \frac{imaginary ( 2 nd Hormonic )}{Real ( 2 nd hormonic )} ) \qquad ( 13 )$$

Similarly, the spherical refractive disorder parameter (Rₛ) and the cylindrical disorder parameter (R_{c}) may be calculated using equations (14) and (15) respectively:

Rₛ=1/2a−abs(b) (14)

R_{c}=2*abs(b) (15)

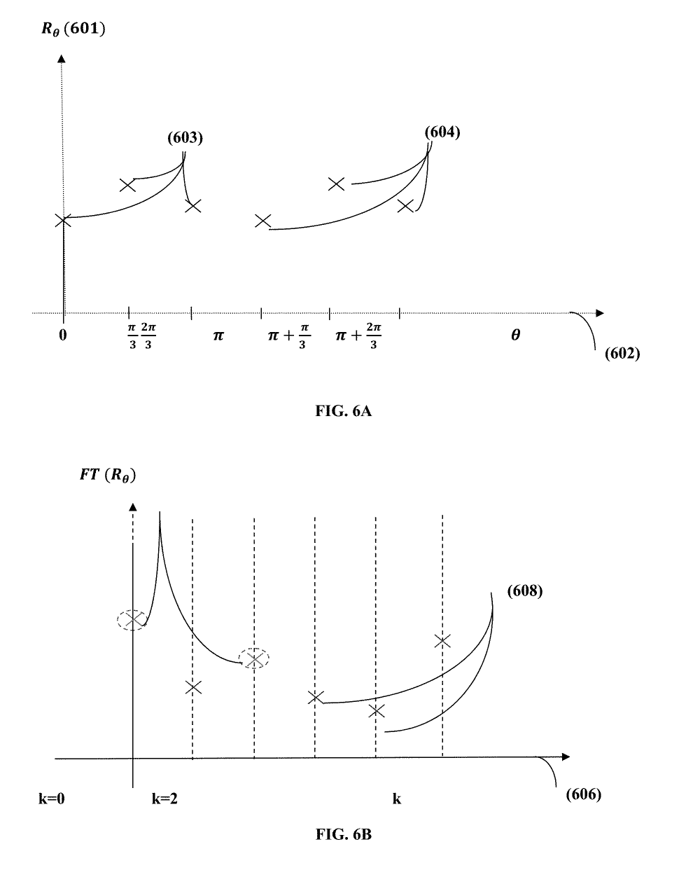

FIG. 6A shows a graphical representation of the Meridian refractive disorder parameter R_{θ} (601) along ‘θ’ axis (602). This represents two sets of points (603) and (604). The point (603) is obtained based on the data array and the point (604) is obtained by duplicating the point (603). The Meridian refractive disorder parameter R_{θ} is calculated as symmetrically located refractive disorder in the angle θ between the angles ‘θ’ to ‘2π’ radians.

FIG. 6B shows the points those need to be considered for obtaining the refractive disorder parameters. The points to be considered for obtaining the refractive disorder parameters may be determined by performing the Fourier transformation on each of sets of points (603) and (604) shown in FIG. 6A.

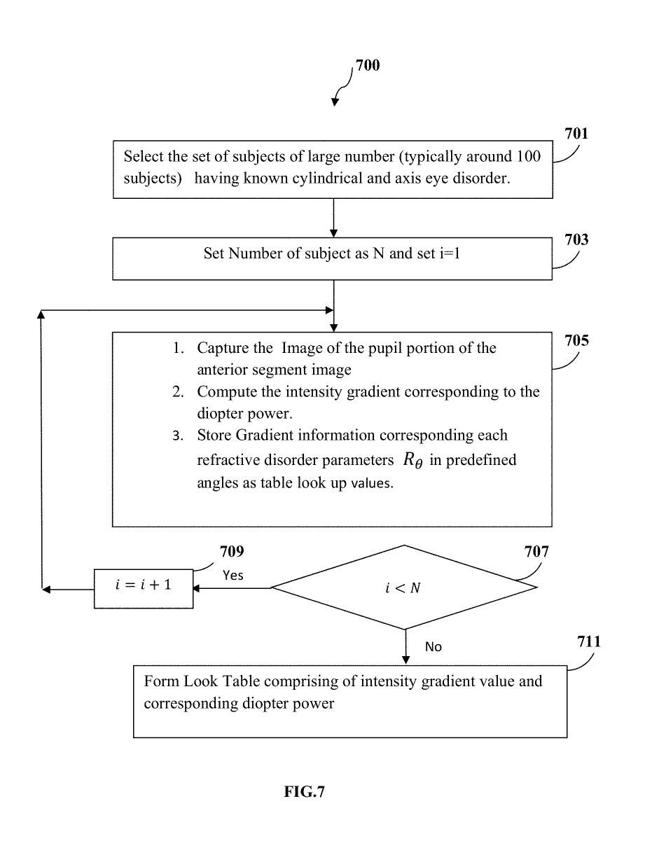

FIG. 7 illustrates a flowchart showing an exemplary method for calibrating the spherical disorder parameter of the eye (208) of the unresponsive object. At block 701, position any eye that does not possess any refractive disorder in front of the image capturing unit (205a) and a gaze is performed at a fixation target object. At block 703, a spherical lens having a predefined optical power is placed in front of the above said normal eye for a period less than 1 second duration. As an example, the predefined optical power of the spherical lens may be +5.0 diopter. At block 705, the image capturing unit (205a) may be used to capture an image of pupil of anterior segment of the normal eye. The captured image may be used to obtain the intensity gradient corresponding to the chosen diopter power by using any one of the strategies (Strategy 1 to Strategy 2) explained earlier. The obtained intensity gradient may be further used to determine the value of the spherical refractive disorder value. At block 707, a lens with a different optical power value is placed in front of the normal eye to calibrate the spherical disorder parameter of the eye. As an example, one or more lenses with an optical power difference factor of 0.25 diopter may be used. At block 709, the selected lens is placed in front of the eye as said in block 707 and the whole process repeated until the spherical disorder parameter of the eye is calibrated.

In an embodiment, as shown in block 711a look up table comprising the spherical disorder parameters and their corresponding slope parameters may be used to analyze the one or more refractive errors of the eye (208a).

### Advantages:

The present disclosure discloses a photo-refraction device for identifying and determining refractive disorder parameters of an eye of an unresponsive subject like a child or an infant and hence diagnosis could be carried out without giving any verbal guiding instructions which makes the process simple and effective.

The optical adapter of the photo-refraction device could be interfaced with any smartphone or PDA (Personal Digital Assistant) for identifying and determining refractive disorder parameters of an eye. Thus the present disclosure eliminates the need for additional or separate components of light sources, and other hardware accessories like camera sensor, processing units, storage systems etc. With the reduction in number of components, it makes the overall design simple and also reduces the cost these additional components would incur.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise. A description of an embodiment with several components in communication with each other does not imply that all such components are required. On the contrary a variety of optional components are described to illustrate the wide variety of possible embodiments of the invention. When a single device or article is described herein, it will be readily apparent that more than one device/article (whether or not they cooperate) may be used in place of a single device/article.

Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERRAL NUMERALS

| Reference Number | Description |
|---|---|
| 101-105 | Flowchart blocks |
| 200 | Photo-refraction device |
| 201 | Computing unit |
| 202 | Optical adapter |
| 203 | Light sources |
| 203a | First LED light source |
| 203b | Second LED light source |
| 203c | Third LED light source |
| 204 | Optical unit |
| 205a | Image capturing unit |
| 205b | Processing unit |
| 205c | Memory unit |
| 206 | Power source |
| 207 | Messaging or E-mail system |
| 208 | Eye of an unresponsive subject |
| 208a | Pupil of the eye |
| 209 | Image plane |

1. A method for capturing images of an eye of an unresponsive subject using a photo-refraction device comprising a computing unit and an optical adapter, the method comprising acts of:
detecting, by an image capturing unit configured in the computing unit of the photo-refraction device, face of the unresponsive subject;
navigating, by processing unit of the computing unit, the image capturing unit towards eye region of the unresponsive subject upon detecting face of the unresponsive subject;
capturing, by the image capturing unit, images of the pupil in real time upon identifying the eye region of the unresponsive subject;
analyzing, by the processing unit, captured images of the pupil to determine feasibility of pupil position;
activating, by the processing unit, a plurality of light sources configured in the optical adapter of the photo-refraction device for sequentially illuminating light on the pupil upon determining the feasibility of pupil position; and
capturing, by the image capturing unit, images of the pupil during sequential illumination of light.

2. The method as claimed in claim 1 comprises an act of controlling, by the computing unit, sequence of illumination of the plurality of light sources.

3. The method as claimed in claim 1, wherein the plurality of light sources is array of Light Emitting Diodes (LEDs).

4. The method as claimed in claim 1 comprises an act of receiving, by an optical unit configured in the optical adapter, irradiant rays of light from the pupil during sequential illumination of light.

5. The method as claimed in claim 4, wherein the optical unit is configured to direct the irradiant rays of light from the pupil onto the image capturing unit.

6. The method as claimed in claim 1, wherein the image capturing unit, captures the images of the irradiant rays of light from the pupil.

7. The method as claimed in claim 1 comprises an act of analyzing, by the computing unit, captured images of the irradiant rays of light from the pupil to determine refractive disorder parameters.

8. The method as claimed in claim 7 comprises an act of communicating, by the computing unit, the refractive disorder to an external device.

9. A photo-refraction device for capturing images of an eye of an unresponsive subject, the device comprising:
an optical adapter comprising a plurality of light sources and an optical unit; and
a computing unit comprising an image capturing unit, a processing unit and a memory unit, the computing unit is interfaced with the optical adapter;
wherein the processing unit is configured to:
detect face of the unresponsive subject via the image capturing unit;
navigate the image capturing unit towards eye region of the unresponsive subject upon detecting face of the unresponsive subject;
receive images of the pupil captured in real time by the image capturing unit upon identifying the eye region of the unresponsive subject;
analyze received images of the pupil to determine feasibility of pupil position;
activate the plurality of light sources configured in the optical adapter for sequentially illuminating light on the pupil upon determining the feasibility of pupil position; and
activate the image capturing unit to capture images of the pupil during sequential illumination of light.

10. The device as claimed in claim 9, wherein the plurality of light sources is array of Light Emitting Diodes (LEDs).

11. The device as claimed in claim 9, wherein the computing unit comprises a power source to supply power to the plurality of LED arrays.

12. The device as claimed in claim 9, wherein the processing unit is configured to process and analyze images obtained by irradiant rays of light from the pupil for measurement of refractive disorder parameters.

13. The device as claimed in claim 9, wherein the memory unit is configured to store captured images of the pupil.

14. A method for determining refractive disorder parameters of an eye of an unresponsive subject, the method comprising:
extracting, by a photo-refraction device, plurality of pixels forming pupil portion of the eye from an image captured using an image capturing device, wherein the plurality of pixels forming the pupil portion of the eye form a feature set;
obtaining, by the photo-refraction device, a gradient image of the pupil portion of the eye by determining direction of each of the plurality of pixels in the feature set;
aligning, by the photo-refraction device, the one or more pixels having similar direction to form one or more lines in the gradient image;
identifying, by the photo-refraction device, a best-fit-line among the one or more lines in the gradient image for obtaining a slope parameter corresponding to the identified best-fit-line; and
assigning, by the photo-refraction device, the slope parameter as the refractive disorder parameter along a meridian axis corresponding to one of a plurality of light sources associated with the image capturing device.

15. The method as claimed in claim 14, wherein the method of extracting the plurality of pixels comprises eliminating one or more redundant pixels in the feature set.

16. The method as claimed in claim 14, wherein the direction of each of the plurality of pixels forming the pupil portion of the eye is one of a vertical direction along the gradient image.

17. The method as claimed in claim 16, wherein the direction of each of the plurality of pixels in the feature set is determined by measuring angle of inclination of each of the plurality of pixels with a reference co-ordinate axis.

18. The method as claimed in claim 14, wherein the best-fit-line is the line joining plurality of correlating pixels among the plurality of pixels in the feature set.

19. The method as claimed in claim 14, wherein the slope parameter corresponding to the best-fit-line is obtained by:
identifying plurality of points on the best-fit-line;
determining a vertical change and a horizontal change in direction of each of the plurality of points on the best-fit-line; and
determining a ratio of difference in vertical change of two of the plurality of points with difference in horizontal change of two of the plurality of points.
